(19) United States
(12) Patent Application Publication
(10) Pub. No.: US 2022/0384031 A1
(43) Pub. Date: Dec. 1, 2022

(54) SYSTEM FOR MONITORING, TRACKING AND RECORDING SAFETY CONDITIONS AND STATUS OF SUBJECTS IN A CONFINED AREA

(71) Applicant: Patrick Chiyin Ng, Swampscott, MA (US)

(72) Inventor: Patrick Chiyin Ng, Swampscott, MA (US)

(21) Appl. No.: 17/329,170

(22) Filed: May 25, 2021

(51) Int. Cl. G16H 40/67; G08B 21/04; G08B 25/00; H04W 12/55

(52) U.S. Cl. CPC G16H 40/67; G08B 21/0407; G08B 21/0469; G08B 25/009; H04W 12/55

(57) ABSTRACT

An integrated system for monitoring, tracking and recording activities of daily livings (ADLs) and safety and wellness conditions of subjects within the same or different locations of a confined area, comprising: alert signal generators, each alert signal generators receives signals from corresponding sensors and/or condition selectable switches disposed in where proximal to each of the subjects residing in the confined area for sensing motions, locations, and change in environmental and/or personal conditions of the corresponding subject or alarm signals actively or passively triggered by the subject, and being activated by said signals from the sensors; corresponding pairing devices physically connected to the alert signal generators for pairing with the corresponding alert signal generators to relay corresponding alert signals to a receiving device, and to receive one or more of responses, information, commands, and/or instructions from the receiving device.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to a system for monitoring, tracking and recording safety conditions and status of subjects in a confined area, and the implementation method thereof.

### BACKGROUND

Aging is one of the challenges most developed and even developing countries are facing given the advance in pharmaceuticals and technologies concerning healthcare for elderly. In particular, the Covid-19 pandemic made a lot of individual seniors stay home without sufficient care and close monitoring of their health and safety conditions. Labor shortage, lack of intensive care staff and special measures imposed in hospitals due to the Covid-19 pandemic also created a lot of potential safety issues in wards where there are not enough caregivers and medical practitioners to cope with the needs.

Some of those residing in elderly home or required to be hospitalized for their remaining lifetime who may suffer from dementia such as Alzheimer's disease, even though proper safety measures/facilities are in place, sometimes could not recall what has happened during the happening of an accident, while the conventional system for monitoring safety conditions or status of individuals requiring intensive care is not sufficient to record and review the event history thereof, which is somehow important to caregivers and medical practitioners in terms of reviewing the current therapeutic regime and intensive care protocol for their patients from time to time to ensure the individuals are well taken care or in good hands.

Therefore, it is of utmost importance to provide an integrated system having an interactive response mechanism and review function of events associated with intensive cares provided by caregivers/medical practitioners to those in need thereof.

### SUMMARY OF THE INVENTION

Accordingly, the present invention provides a system and implementation method for monitoring, tracking and recording safety and wellness conditions of subjects within the same or different locations of a confined area such that caregivers, medical practitioners, and even family members of the subjects can more easily track the conditions and status of the subjects within a larger confined area with a significant number of subjects to be looked after and closely monitored, while the event history, e.g., Activities of Daily Living (ADLs), accident such as a fall or lost, can be more easily retrieved and reviewed than the conventional system for the same or similar purpose.

In one aspect, the system of the present invention includes:

one or more alert signal generators, each of the alert signal generators receiving signals from one or more corresponding sensors and/or condition selectable switches disposed in where proximal to each of the subjects residing in the confined area for sensing motions, locations, and change in environmental and/or personal conditions of the corresponding subject or alarm signals actively or passively triggered by the subject, and being activated by said signals from the sensors;

one or more corresponding pairing devices physically connected to the one or more alert signal generators for pairing with the corresponding alert signal generators to relay corresponding alert signals of the sensed motion, change in environmental conditions, and/or personal conditions by the corresponding sensors or by the condition selectable switches to a receiving device, and to receive one or more of responses, information, commands, and/or instructions from the receiving device;

one or more receiving devices, each of the receiving devices including at least an alert signal transceiver and a processor, to receive the alert signals from the one or more corresponding pairing devices, directly transmit the alert signals to one or more internal or external devices with respect to the system, or process the alert signals from the one or more pairing devices to generate alert data to transmit to said internal or external devices, receive one or more of signals, responses, information, commands, and/or instructions from the one or more internal or external devices, and relay the one or more of signals, responses, information and/or instructions from the one or more internal or external devices to the one or more signal generators or to other internal or external devices;

said internal or external devices responding to said alert signals or data, and one of the internal or external devices assigning an identity and location data to each of the subjects after receiving the alert signals directly from the one or more pairing devices through the one or more receiving devices or the alert data from the one or more receiving devices such that each set of alert signals or data is attached with said identity and location data assigned by said one of the internal or external devices;

the alert signals or data from said receiving devices and one or more of signals, responses, information and/or instructions from said internal or external devices being timestamped, recorded and stored in an internal or external storage in order for being retrieved and reviewed by caregivers and/or medical practitioners for said subjects when in need thereof.

Another aspect of the present invention provides a method for monitoring, tracking and recording safety status and conditions of a subject under intensive care by caregivers and/or medical practitioners using the present system described herein, the method comprising:

disposing a plurality of sensors and/or condition selectable switches in where proximal to the subject residing in a confined area;

connecting said plurality of sensors and/or condition selectable switches to an alert signal generator device;

connecting a corresponding pairing device to said alert signal generator;

pairing said pairing device to said alert signal generator;

connecting said pairing device to a receiving device which is connected to a first internal or external device which monitors connection and operational statuses of said sensors, condition selectable switches, alert signal generator, and/or pairing device;

said receiving device further connecting to a second and/or subsequent internal or external devices being used by said caregivers and/or medical practitioners;

when one of the sensors/condition selectable switches sends a signal to the alert signal generator due to a change in motion, temperature, proximity, moisture, pressure, gas level, and any other environmental or personal data, the paired pairing device sends an alert signal data to the receiving device before relaying thereof to different internal or external devices connected to said receiving device;

once the second or subsequent internal or external device sends a confirmation notification or message regarding attendance to an event associated with the alert signal data to the receiving device, other internal or external devices will receive a message from the receiving device that the event is attended;

one or more of the alert signal data, change of setting in said sensors, condition selectable switches, alert signal generator device, corresponding pairing device, second and/or subsequent internal or external devices, the attendances of different events associated with the alert signal data, feedback signal from one or more internal or external devices to the change of setting in different devices of the system and/or to the attendances of different events is/are recorded and stored in either local or external memory of the system which is accessible and retrievable by the first internal or external device.

Other features, embodiments and variations of the present system and methods of implementing the system for monitoring, tracking and recording the safety status and conditions of subject in need of the system are set forth in the following Detailed Description of the Invention.

### BRIEF DESCRIPTION OF THE DRAWINGS

Embodiments of the invention are described in more details hereinafter with reference to the drawings, in which:

FIG. 1A schematically depicts from a perspective view an alert signal generator device according to an embodiment of the present invention;

FIG. 1B schematically depicts from a front view a pairing device with a wireless convertor device according to an embodiment of the present invention;

FIG. 1C schematically depicts from a perspective view an alert signal generator device which is connected to a pairing device with a wireless convertor device according to an embodiment of the present invention;

FIG. 2A is a schematic block diagram of the components of an alert signal generator device according to an embodiment of the present invention;

FIG. 2B is a schematic block diagram of the circuitry of one of the remote wireless convertor devices according to an embodiment of the present invention;

FIG. 3A generally depicts the interaction between different key components of the present system;

FIG. 3B schematically depicts a workflow of how the present system sends and processes signal data from different sensors/condition selectable switches with at least two levels of receiving devices according to an embodiment of the present invention;

FIG. 3C schematically depicts how different internal/external devices are controlled and monitored by a first internal/external device through two levels of receiving devices according to an embodiment of the present invention;

FIG. 4A illustrates a workflow of how a pairing device is triggered according to an embodiment of the present invention;

FIG. 4B illustrate a workflow of how a pairing device receives and processes a change of setting according to an embodiment of the present invention;

FIG. 4C illustrates a workflow of how a pairing device manages a low battery situation according to an embodiment of the present invention;

FIG. 4D illustrates a workflow of how to check the alive status of a pairing device;

FIG. 5A illustrates a workflow of how a pager receives and processes an alert signal from the receiving device according to an embodiment of the present invention;

FIG. 5B illustrates a workflow of how to change setting of a pager according to an embodiment of the present invention;

FIG. 5C illustrates a workflow of how to charge a pager's power according to an embodiment of the present invention;

FIG. 5D illustrates a workflow of how to manage a lower battery situation of a pager according to an embodiment of the present invention;

FIG. 5E illustrates a workflow of how to check the alive status of a pager according to an embodiment of the present invention;

FIG. 6A illustrates a workflow of how to check associated device status by a receiving device according to an embodiment of the present invention;

FIG. 6B illustrates a workflow of how to check power type of a receiving device by a first internal/external device according to an embodiment of the present invention;

FIG. 6C illustrates a workflow of how to detect and synchronize alert data with a second/subsequent receiving device according to an embodiment of the present invention;

FIG. 6D illustrates a workflow of how a receiving device receives signal from a paired pairing device according to an embodiment of the present invention;

FIG. 6E illustrates a workflow of how a receiving device receives and processes a confirmation notification/alert message from a pager according to an embodiment of the present invention;

FIG. 7A illustrates a workflow of how a primary internal/external device changes setting of a connected pairing device according to an embodiment of the present invention;

FIG. 7B illustrates a workflow of how a primary internal/external device changes the setting of a second or subsequent internal/external device according to an embodiment of the present invention;

FIG. 8A schematically depicts an implementation embodiment of the present system in a one-room environment; and

FIG. 8B schematically depicts another implementation embodiment of the present system in a two-room environment.

### DETAILED DESCRIPTION OF THE INVENTION

In the following description, methods and systems for monitoring, tracking and recording safety conditions and status of subjects in confined areas are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

It should be apparent to practitioner skilled in the art that the foregoing examples of digital driving methods are only for the purposes of illustration of working principle of the present invention. It is not intended to be exhaustive or to limit the invention to the precise forms disclosed.

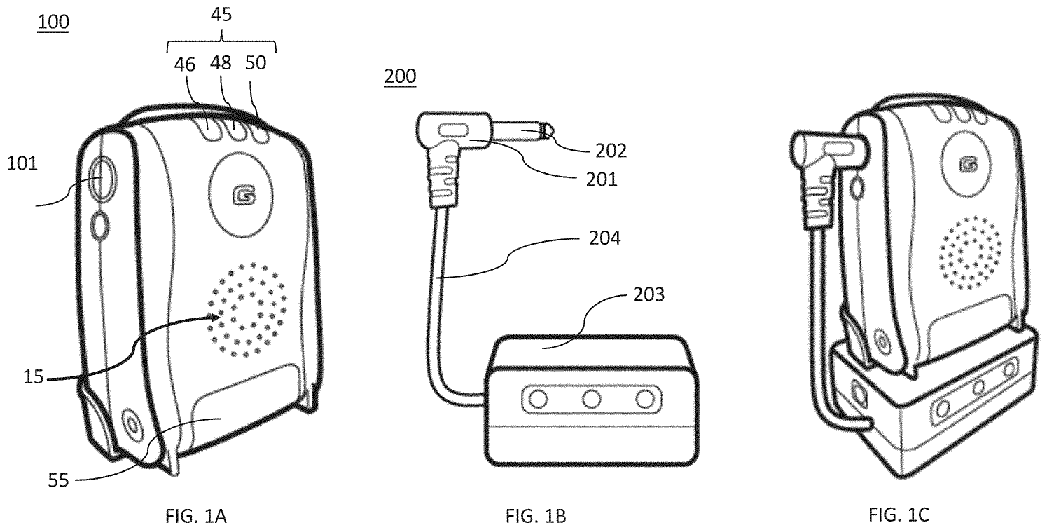

Turning to FIG. 1A, an alert signal generator device 100 includes a plurality of light emitting diode (LED) internal status indicators (46, 48, 50) on top of a case, a speaker 15 for audible output signal, an LED array 55 at the bottom of the case including a plurality of remote status indicators (not shown clearly in this figure) for indicating the pairing status/operational condition of the paired sensors/condition selectable switches, and a slot 101 for a connector pin 202 of a corresponding dongle 201 as shown in FIG. 1B.

Turning to FIG. 1B, a pairing device 200 includes a dongle 201 and a wireless convertor 203 connected through a wire or cable 204 with each other, and the dongle 201 includes a connector pin 202 for plugging into the corresponding slot 101 on the case of the alert signal generator device 100 in order to relay the alert signals generated by the alert signal generator and also location data of the corresponding sensors/condition selectable switches to other device(s) which has/have been paired with the pairing device. The basic working principle is that when the alert signal generator is connected with the dongle device (FIG. 1C), and is triggered by signals from the corresponding sensors/condition selectable switches, an alert signal and the location data of the dongle device are sent wirelessly through the connected dongle device to a paired receiving device (not shown in these figures), and then the receiving device communicates for such alert signals/location data with other paired devices such as pagers or mobile devices of the corresponding caregivers and/or medical practitioners. A “pairing device” is any device passive (for example: motion sensor and etc.) or active (for example: emergency call button and etc.).

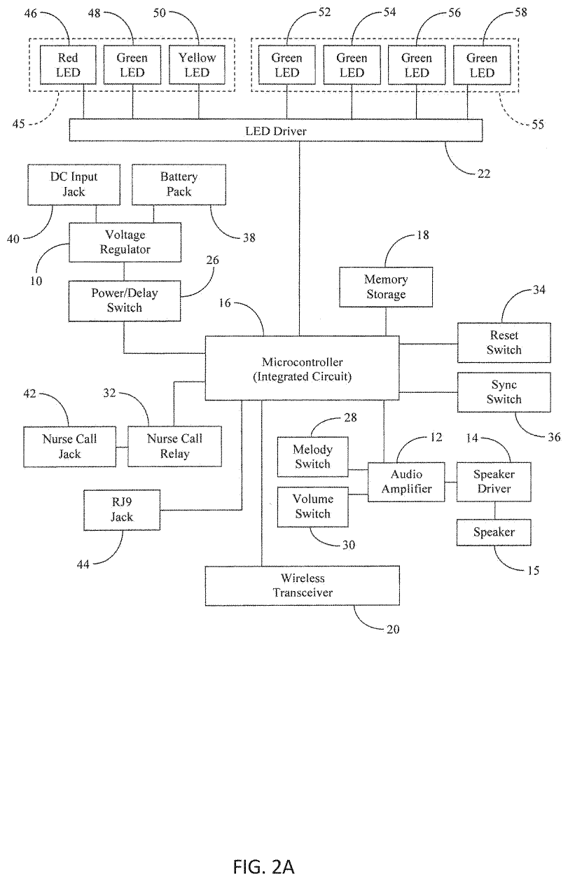

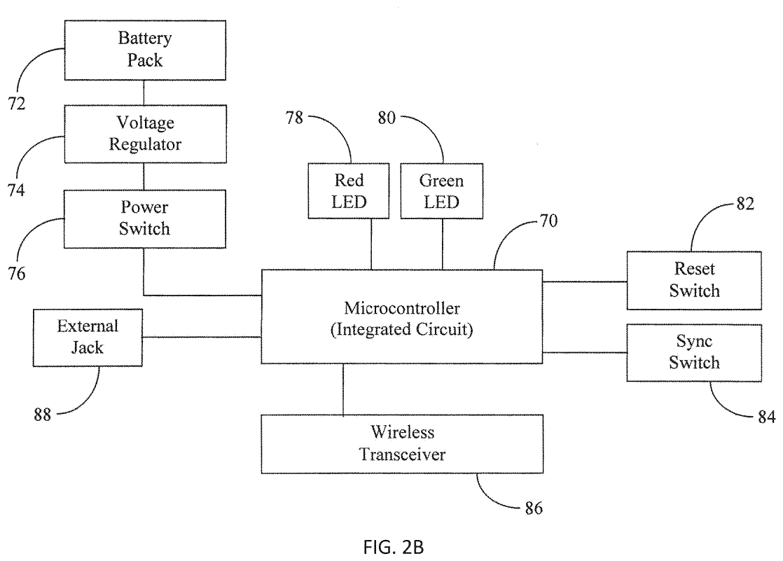

FIG. 2A illustrates a block diagram of key components of an alert signal generator device according to an embodiment of the present invention. The alert signal generator device includes a microcontroller 16, a wireless transceiver 20, a power supply (in this example, a battery pack) 38, at least one audio output component 15, a first LED array 45 including a plurality of internal status indicators (46, 48, 50), and optionally a second LED array 55 including a plurality of remote status indicators (52, 54, 56, 58). The first LED array 45 indicates the status of the alert signal generator device while the second LED array 55 indicates the pairing status of different sensors and/or condition selectable switches with the alert signal generator device. In other embodiment, the alert signal generator device is optionally connected to an external nurse call relay 32 and nurse call jack 42 for monitoring multiple subjects in different locations within a confined area. In another embodiment, the alert signal generator device is optionally connected to some external devices such as wireless (or cellular) notification and alarm systems via a communication port 44. Alert signal data could be temporarily stored in the memory 18 or stored/synchronized with an external memory to the present system. The size and structure of the alert signal generator device are variable depending on ease of installation, setup and operation, etc. The visual and audible signaling components provide at-a-glance information on the status of the device (system) and any problems requiring immediate attention. The alert signal generator device is also incorporated with synchronization function to pair with the corresponding sensors and/or condition selectable switches, and a lost signal feature to ensure that the paired sensors and/or condition selectable switches are in operation within certain area over a period of time.

FIG. 2B illustrates a block diagram of key components of a wireless convertor device according to an embodiment of the present invention. The wireless convertor device includes a microcontroller 70, a wireless transceiver 86, a power supply (in this example, a battery pack) 72, and an external jack 88 for hardwire connection to the associated sensors and/or condition selectable switches. The size and structure of the wireless convertor device are also variable depending on the ease of placement, connection to corresponding sensors/condition selectable switches, and operation. Some of the wireless convertor devices may have adhesive surfaces to position next to the sensors/condition selectable switch.

In other embodiment, because some sensors and/or condition selectable switches may be incorporated with wireless communication circuitry, and therefore the wireless convertor could be omitted in the present system. Pairing may occur as in the manner of the wireless convertor, or may require additional steps dependent on the pairing procedures specified by the sensors/condition selectable switches.

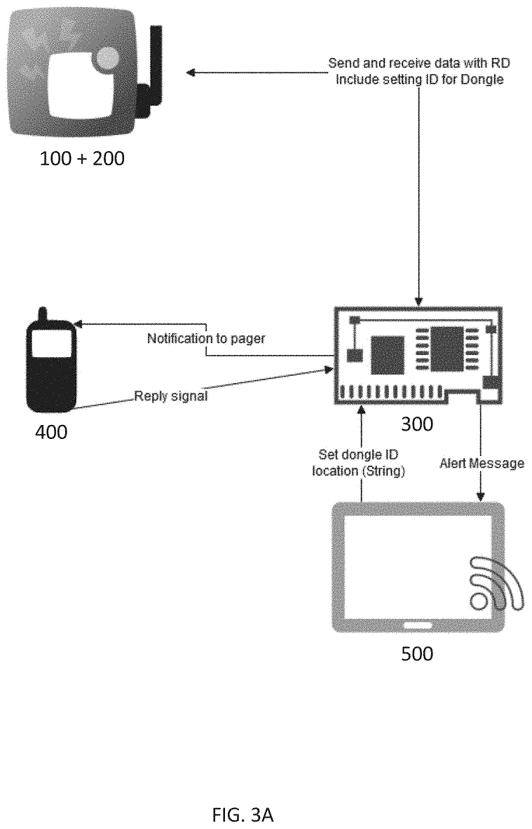

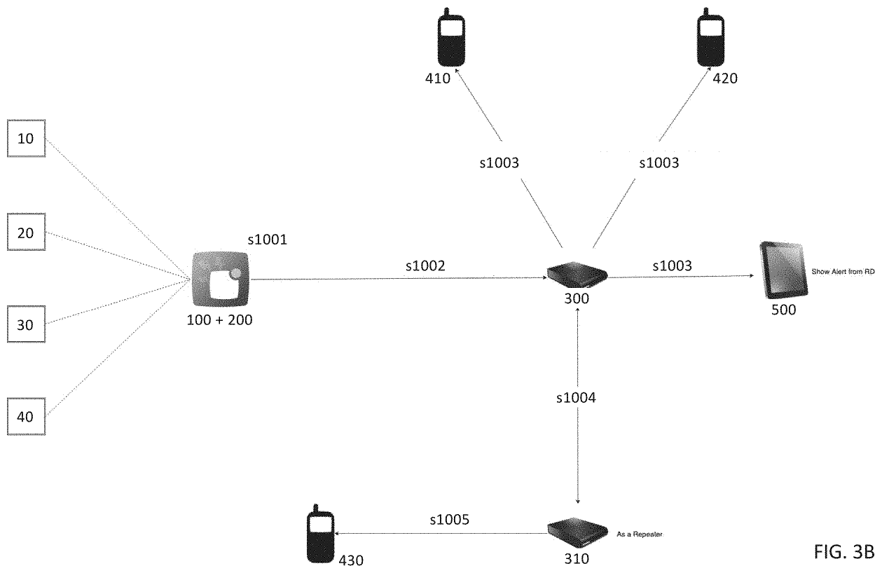

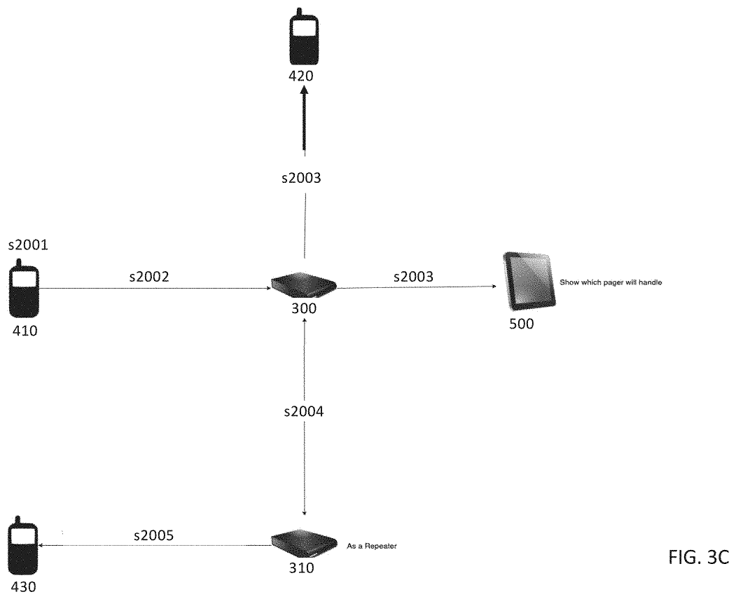

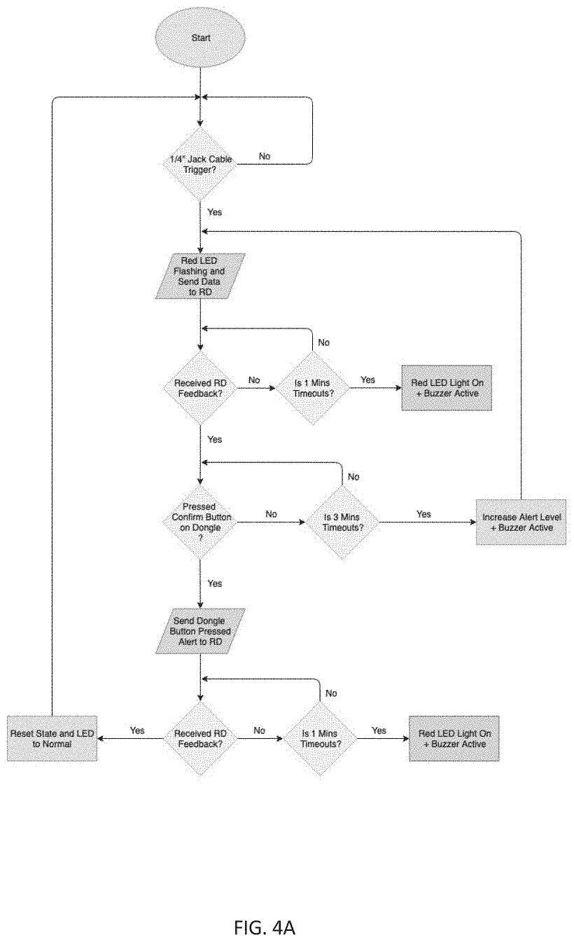

Turning to FIG. 3A, once the alert signal generator device 100 and pairing device 200 are connected and paired, and the signal transceiver in the wireless convertor can send signal data to the corresponding receiving device 300, where the receiving device 300 may directly transmit the signal data from the pairing device 200 to a corresponding pager (or a smartphone) 400 and mobile device 500, or process the signal data from the pairing device 200 before transmitting a notification or alert message to any of the pager/smartphone 400 and mobile device 500. The mobile device 500 can send an instruction to assign an identity and change setting of the pairing device 200 through the receiving device 300. The page 400 can also send a reply to the pairing device 200 through the receiving device 300 such as the alert message is read and attended.

Turning to FIG. 3B, it illustrates how alert data is sent from the sensors/condition selectable switches according to the present invention is as follows:

s1001: after the alert signal generator device 100 receiving signals from different sensors/condition selectable switches (10, 20, 30, 40), and successfully connected and paired with the corresponding pairing device 200, the alert signal generator device 100 is activated to trigger the pairing device 200 to send out the alert data;
s1002: alert data including the name, identity, and location of the subject is sent to a first receiving device 300;
s1003: the first receiving device 300 relays the alert data from the pairing device 200 to a first and second pagers or smartphones (410, 420) and mobile device 500;
s1004: the first receiving device 300 also relays the alert data to a second receiving device 310 where the second receiving device 310 serves as a “repeater” of the first receiving device 300;
s1005: the second receiving device 310 repeats the alert data from the first receiving device 300 and alert data including name and location of the subject to a third pager or smartphone 430.

Turning to FIG. 3C, it illustrates how data is sent from one pager/smartphone and communicate such data with other pagers/smartphones/mobile device within the present system in the case where a caregiver/medical practitioner tries to communicate with other caregivers/medical practitioners an event associated with a received alert signal/data is to be handled:

s2001: caregiver/medical practitioner of a first pager or smartphone 410 sends a confirmation notification by pressing a button or alike on the first pager to communicate with other internal or external devices paired with the present system that the event associated with the received alert signal/data;
s2002: the confirmation notification from the first pager or smartphone 410 is sent as a pager or smartphone handle data to a first receiving device 300;
s2003: the first receiving device 300 relays the pager or smartphone handle data to a second pager or smartphone 420 and a mobile device 500 paired with the first receiving device 300;
s2004: the first receiving device 300 also sends the same pager or smartphone handle data to a second receiving device 310;
s2005: the second receiving device 310 repeats the same pager or smartphone handle data from the first receiving device 300 and sends the same to a third pager or smartphone 430 paired with the second receiving device 310.

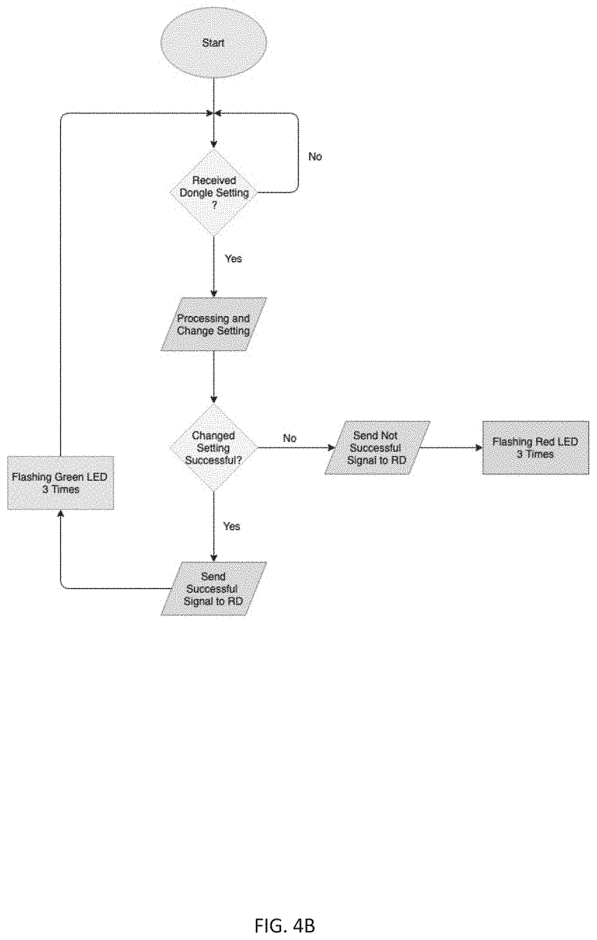

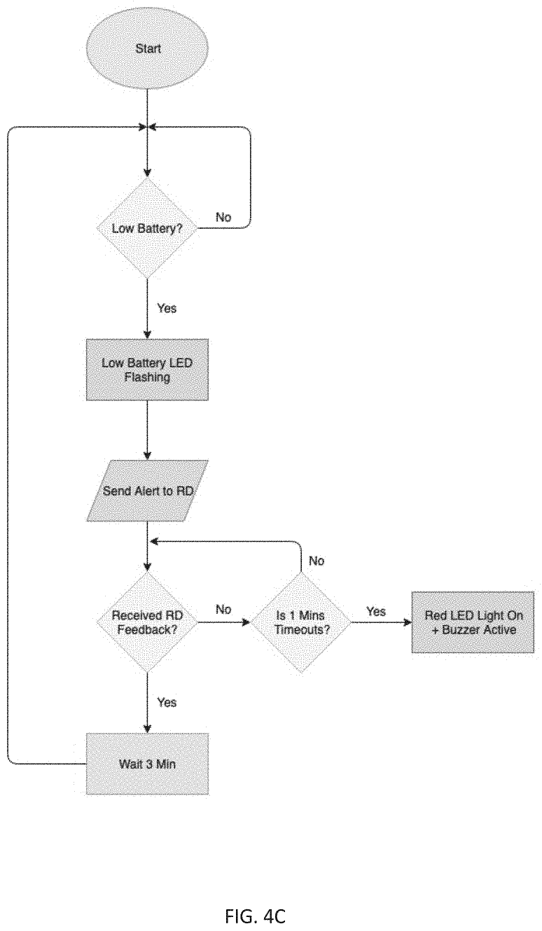

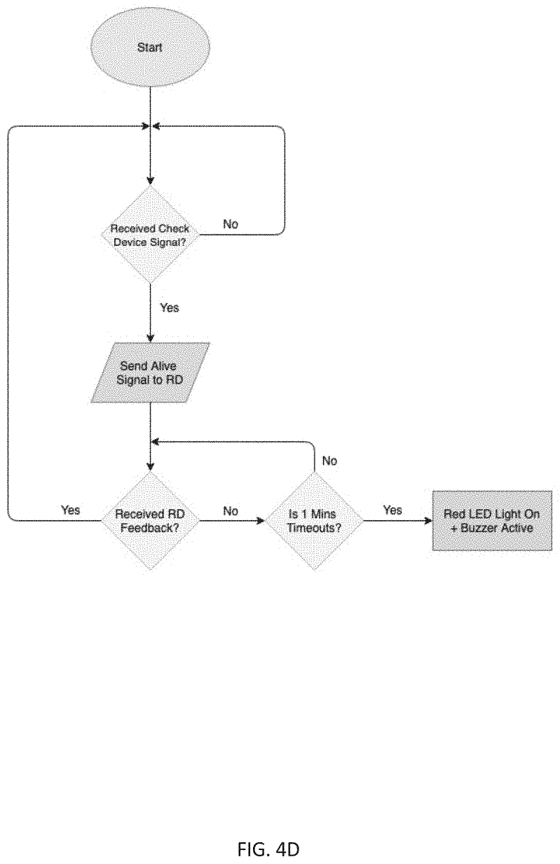

Turning to FIG. 4A, the workflow diagram illustrates how a pairing device is activated. First, if the connection pin, e.g., ¼″ jack cable, of the pairing device is connected to the corresponding slot on the case of the alert signal generator, a red LED indicating the pairing device is physically connected and paired to the alert signal generator flashes, and followed by sending a data to the corresponding receiving device. If the receiving device sends a feedback signal to the pairing device, the user of the pairing device should press a confirm button on the pairing device to confirm a successful pairing to the corresponding receiving device. On the other hand, if the feedback from the receiving device is not received within certain period of time, e.g., 1 minute, the red LED turns solidly on with a buzzer turned on. Once the user has confirmed the receipt of the receiving device's feedback, a pairing device button pressed alert will be sent to the receiving device. Otherwise, if the confirm button on the pairing device is not pressed within certain period of time, e.g., 3 minutes, the alert level will be elevated accompanied with a buzzer turned on. The receiving device will further feedback to the pairing device button pressed alert and if such feedback is received by the pairing device, the state of the pairing device will be reset and the LED indicator will resume normal. In the case where any feedback from the receiving device is not received, after certain period of time, e.g., 1 minute, the red LED indicator on the pairing device will be “on” accompanied with a buzzer sound.

FIG. 4B illustrates a workflow of how a pairing device receives and processes a change of setting according to an embodiment of the present invention. The pairing device receives and processes a pairing device setting change. If the setting change is successful, a successful signal is sent to the receiving device; and a green LED flashes 3 times. However, if the setting change is unsuccessful, an unsuccessful signal is sent to the receiving device; and a red LED flashes 3 times.

FIG. 4C illustrates a workflow of how a pairing device manages a low battery situation according to an embodiment of the present invention. The pairing device detects low battery. A low battery LED flashes, and followed by sending an alert to the receiving device. If the receiving device sends a feedback signal to the pairing device, the pairing device waits for certain period of time, e.g., 3 minutes, and reiterates from the beginning of the workflow. On the other hand, if the feedback from the receiving device is not received within certain period of time, e.g., 1 minute, a red LED turns solidly on with a buzzer turned on.

FIG. 4D illustrates a workflow of how to check the alive status of a pairing device. The pairing device is set to receive a Check Device signal corresponding to its alive status; and if a Check Device is received, it sends an Alive signal to the receiving device. If the receiving device sends a feedback signal to the pairing device, the pairing device reiterates from the beginning of the workflow. On the other hand, if the feedback from the receiving device is not received within certain period of time, e.g., 1 minute, a red LED turns solidly on with a buzzer turned on.

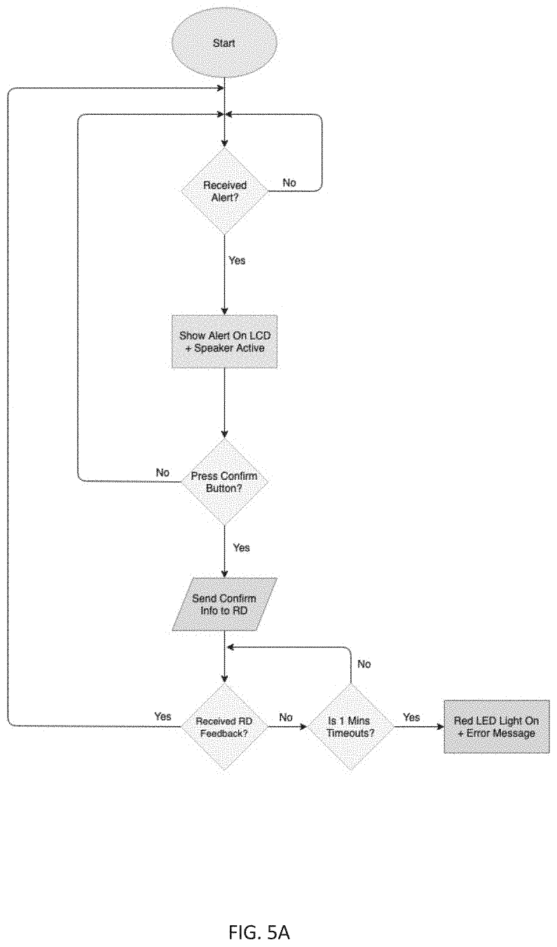

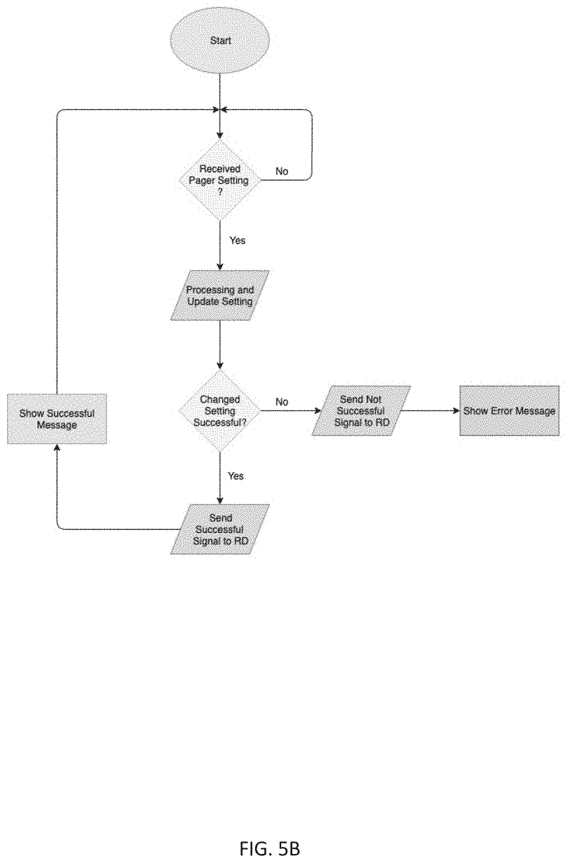

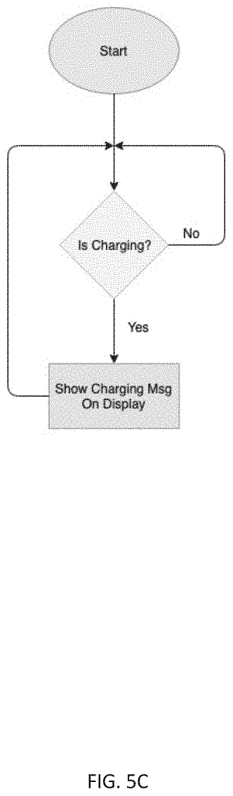

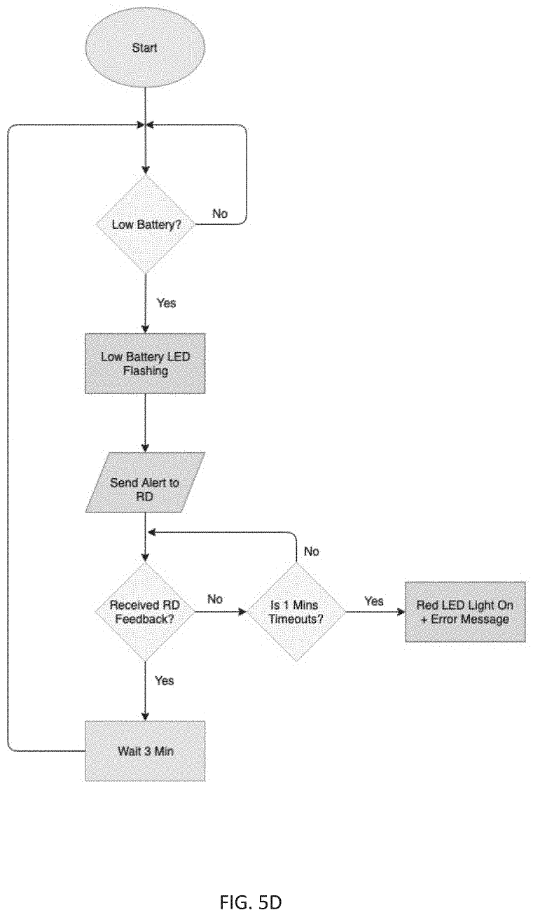

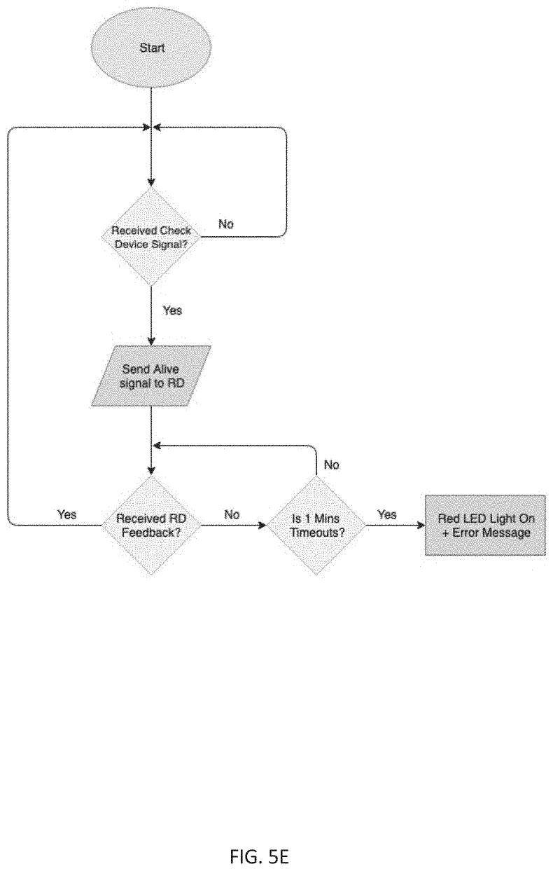

Turning to FIG. 5A, the workflow illustrates how pager reacts to an alert signal/data from the receiving device. When a pager receives an alert signal/data from the receiving device, an alert message is shown on a display of the pager with an alert sound if the speaker thereof is activated. The user of the pager is asked to confirm receipt of the alert message. After a while, if the user has confirmed receipt of the alert message by pressing a button or alike on pager to confirm, a confirmation signal will be sent back to the receiving device. The receiving device will then send a feedback signal to the pager confirming receipt of the confirmation signal from the pager. If the user of the pager does not confirm receipt of the alert message, the alert message together with the alert sound will keep going on the pager until the user confirms receipt thereof. If the receiving device does not send a feedback signal confirming receipt of the confirmation signal from the pager, after certain period of time, e.g., 1 minute, a red LED indicator on the pager will be “on” with an error message informing the unsuccessful receiving of the confirmation signal by the receiving device.

FIG. 5B illustrates a workflow of how a pager device receives and processes a change of setting according to an embodiment of the present invention. The pager receives and processes a pager setting change. If the setting change is successful, a successful signal is sent to the receiving device, and the pager displays an update successful message. However, if the setting change is unsuccessful, an unsuccessful signal is sent to the receiving device; and the pager displays an update successful message.

FIG. 5C illustrates a workflow of how to charge a pager's power according to an embodiment of the present invention. The pager detects a charging and displays a charging message on its display.

FIG. 5D illustrates a workflow of how to manage a lower battery situation of a pager according to an embodiment of the present invention. When the pager detects a low battery condition, a low battery LED flashes, and an alert is sent to the receiving device. If the pager receives from the receiving device a feedback signal, the pager reiterates from the beginning of the workflow after waiting for period of time, e.g., 3 minutes. On the other hand, if the feedback from the receiving device is not received within certain period of time, e.g., 1 minute, a red LED turns solidly on with an error message displayed.

FIG. 5E illustrates a workflow of how to check the alive status of a pager according to an embodiment of the present invention. The pager is set to receive a Check Device signal corresponding to its alive status; and if a Check Device is received, it sends an Alive signal to the receiving device. If the receiving device sends a feedback signal to the pager, the pager reiterates from the beginning of the workflow. On the other hand, if the feedback from the receiving device is not received within certain period of time, e.g., 1 minute, a red LED turns solidly on with an error message displayed.

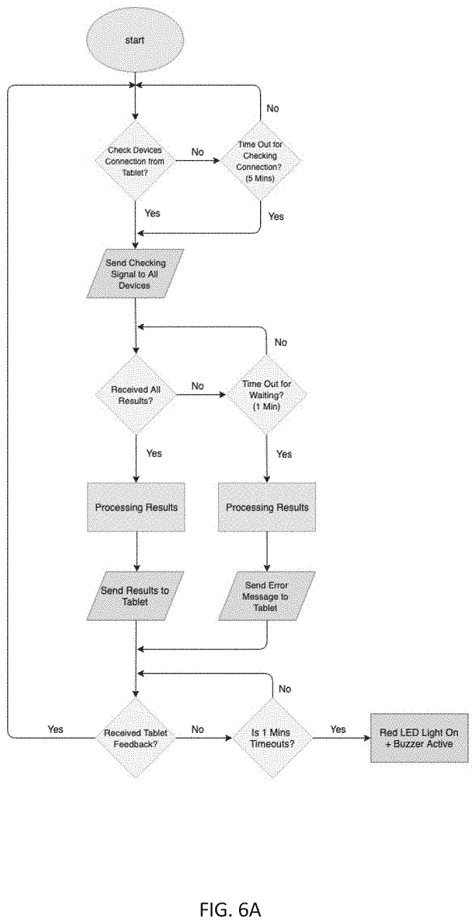

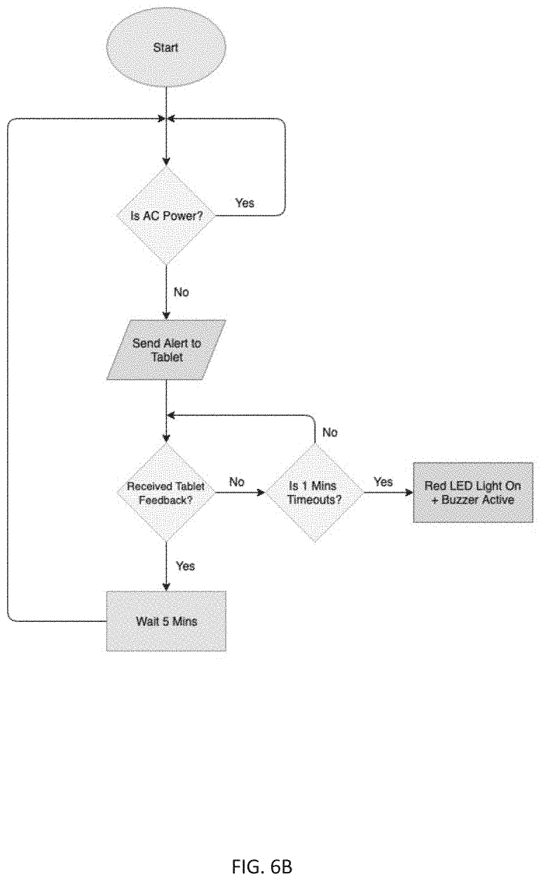

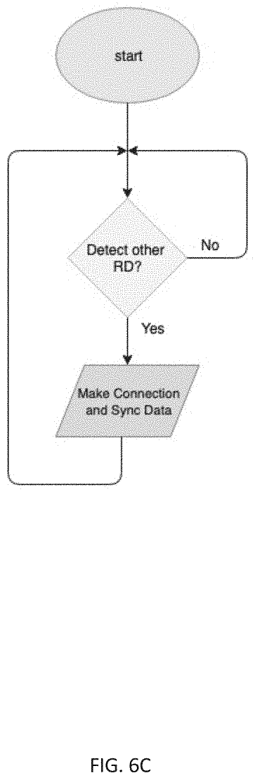

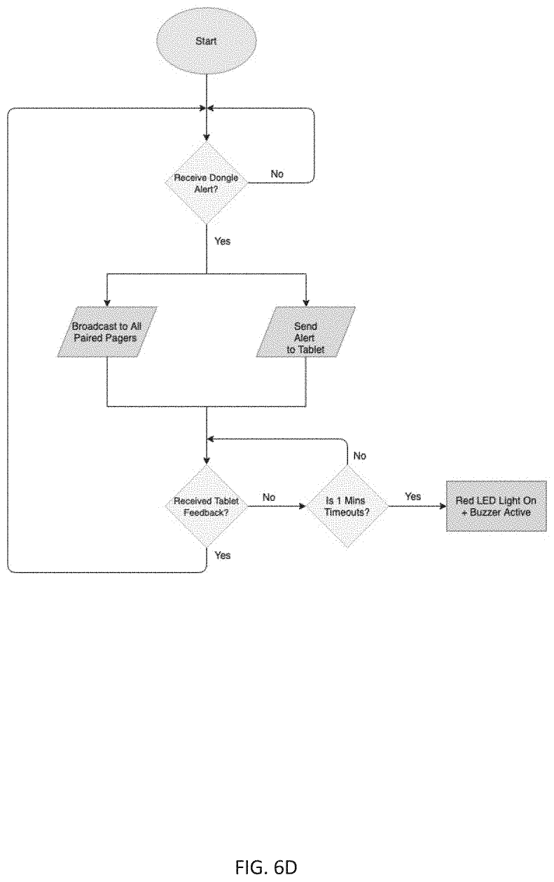

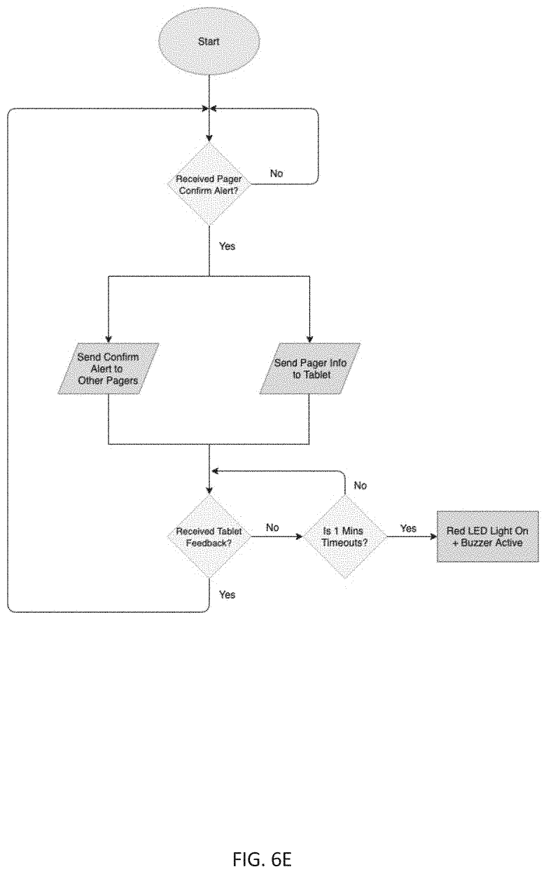

Turning to FIG. 6A, the workflow illustrates how a receiving device checks the connection status of the corresponding devices paired to the receiving device by a paired external device, in this example, is a tablet. Once the tablet is paired with the receiving device, connection status of the corresponding devices such as pairing device, pagers, a second receiving device, etc., should be checked by the user of the tablet first before use. If the connection status of the corresponding devices to the receiving device is not checked, after a while, a request for checking the connection status thereof will be sent to the tablet. Once the connection status of the corresponding devices to the receiving device is checked, a checking signal will be sent to all corresponding devices connected to the receiving device. The tablet will also receive results from all corresponding devices through the receiving device in order to process the results. However, after a while, if the tablet does not receive the results from the corresponding devices through the receiving device, an error message will be shown on a display of the tablet. Once the tablet has confirmed receipt of the results from the corresponding devices, the tablet will send a feedback signal to all the corresponding devices confirming receipt of the results.

Turning to FIG. 6B, the workflow illustrates how to check the power type of a receiving device by a first internal/external device. If the receiving device detects a disconnection from an AC power source, an Alert sign is sent to the tablet. If the tablet sends a feedback signal to the receiving device, the receiving device reiterates from the beginning of the workflow after waiting for a period of time, e.g., 5 minutes. On the other hand, if the feedback from the tablet is not received within certain period of time, e.g., 1 minute, a red LED turns solidly on with a buzzer turned on.

Turning to FIG. 6C, the workflow illustrates how a first receiving device detects a second receiving device to relay signal data from the corresponding pairing device. When the first receiving device detects a second receiving device requesting for pairing, the first receiving device will connect to the second receiving device and synchronize data received from the corresponding pairing device such that the second receiving device will repeat the signal data from the first receiving device when the second receiving device sends the same to one or more corresponding internal/external devices paired thereto, e.g., a second or subsequent pager which is paired with the first receiving device.

Turning to FIG. 6D, the workflow illustrates how the receiving device receives alert data from the pairing device. When the receiving device receives alert signal from a paired pairing device, it will relay the alert signal data to all the other paired external devices such as pagers and mobile device. In the case of a tablet monitoring the receiving device, the receiving device will send an alert message to the tablet. The tablet will send a feedback signal to the receiving device and further relay to the pairing device. If the feedback signal is not received by the receiving device, after a while (e.g., 1 minute), a feedback signal will be re-sent by the tablet. On the other hand, the red LED indicator will be “on” and a buzzer sound will be generated when the feedback signal from the tablet is received by the receiving device.

Turning to FIG. 6E, the workflow illustrates how the receiving device receives and processes the confirmation notification/alert from the pager confirming the attendance to the event associated with the alert data. When the receiving device receives a confirmation notification/alert data regarding the attendance to the event associated with the alert data confirmed by the user of a first pager, the receiving device will send a confirmation alert to other pagers paired with the receiving device and also send the corresponding pager information to the tablet monitoring the receiving device. The tablet will send a feedback signal to the receiving data. Once the feedback signal is received, the receiving device will further relay such a feedback signal to the corresponding pager. What if the feedback signal is not received by the receiving device, a timer will count for a certain period of time, e.g., 1 minute. If, after said certain period of time, the receiving device still does not receive the feedback signal from the tablet, a red LED indicator will be “on” accompanied with a buzzer sound.

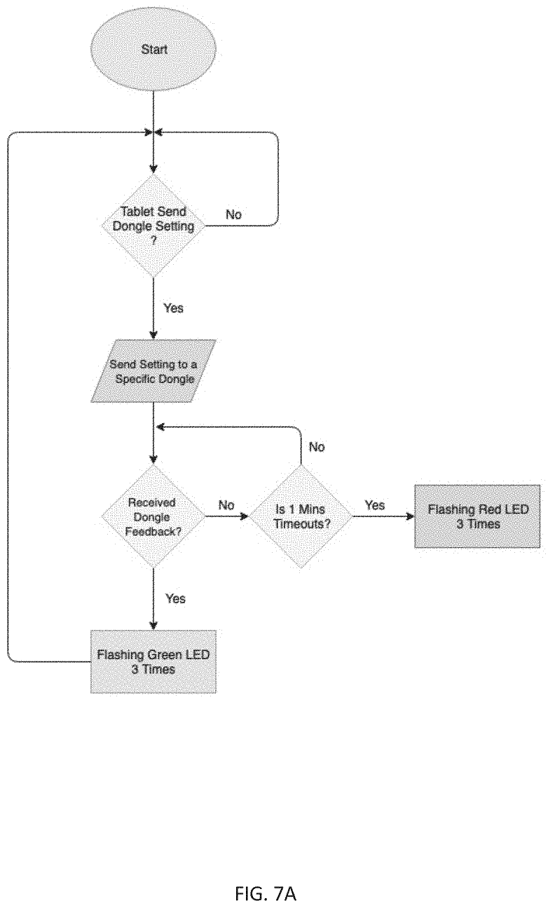

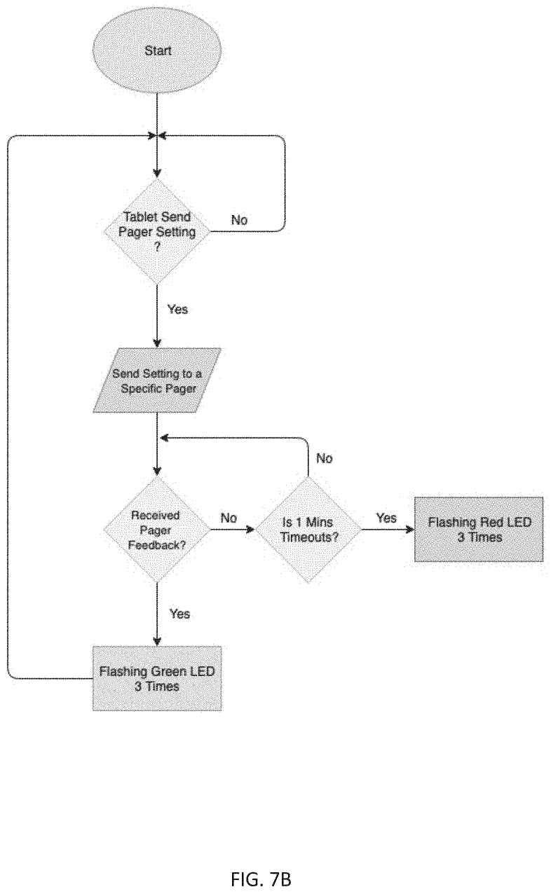

Turning to FIG. 7A, the workflow illustrates how a mobile device, e.g., a tablet, changes setting of a specific pairing device. It is assumed that the pairing device is configured to send and receive signal wirelessly to and from the tablet directly in this example. It is possible that an intermediate device such as the receiving device of the present invention is incorporated into this workflow in the case where the pairing device is not directly connected to the tablet or requires the intermediate device to communicate with the tablet. When the tablet sends a pairing device setting request to a specific pairing device, and received by the specific pairing device, the specific pairing device will send a feedback signal to the tablet. Once the feedback signal is received, the green LED indicator will flash not just once, e.g., for three times. If the feedback signal is not received from the specific pairing device, a timer will count for a certain period of time, e.g., 1 minute. If, after said certain period of time, the feedback signal is still not received by the tablet, it will request the specific pairing device to send the feedback signal again. What if the feedback signal is received after said certain period of time, a red LED indicator will flash not just once, e.g., three times.

Turning to FIG. 7B, the workflow illustrates how a tablet changes setting of a pager. Similar to the workflow as shown in FIG. 7A, the tablet sends a request for change of setting to a specific pager, and if the pager does not send a feedback signal to the tablet, after a certain period of time (e.g., 1 minute) counted by a timer, the tablet will request the corresponding pager to send a feedback signal again. On the other hand, if the feedback signal is sent and received by the tablet after said certain period of time, a red LED indicator will flash not just once, e.g., three times. It should be noted that in certain embodiments of the present invention, pager is substitutable by any mobile devices including smartphone, if appropriate.

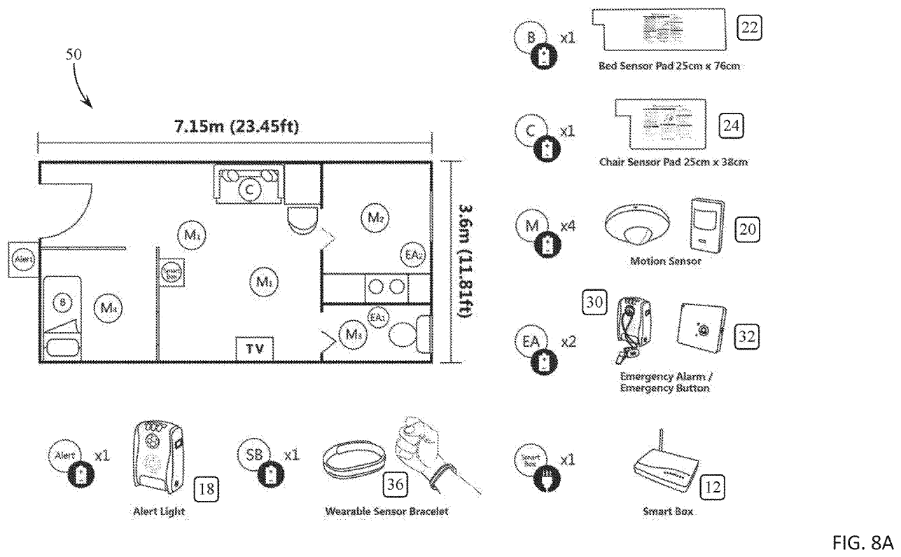

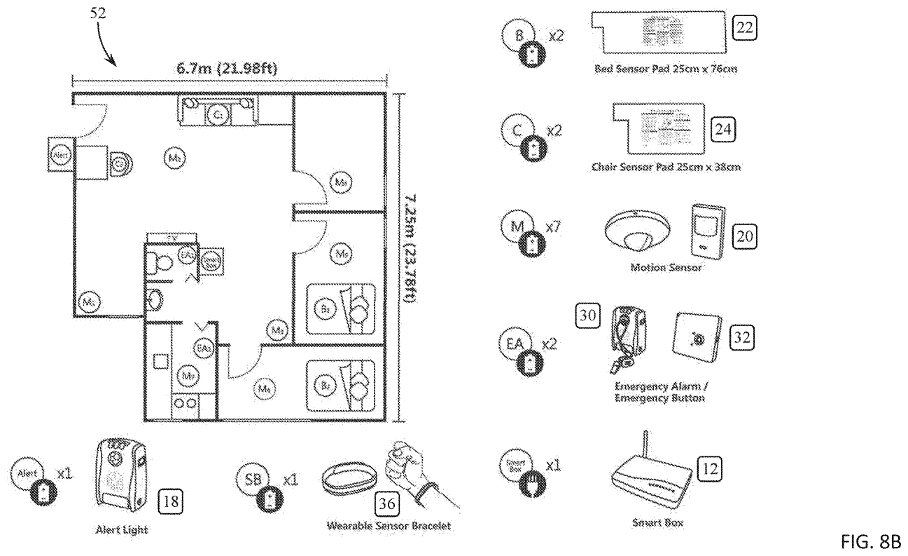

Turning to FIG. 8A, it shows a one-room environment installed with one bed sensor 22, one chair sensor 24, four motion sensors 20, two emergency pull cord 30, and/or push button 32, a system gateway 12 (a primary communication unit which may be physically connected to an external internet network via wires/fiber optics and communicates internally with all sensors/condition selectable switches or signal convertors such as wireless signal convertor), a wearable sensor bracelet 36, and an alert light 18. In accordance to another embodiment, the wearable sensor bracelet 36 can be substituted with another form of wearable device readily realizable by a skilled person in the art including, but not limited to, a sensor-embedded jewelry and a keyring sensor.

Turning to FIG. 8B, it shows a two-room environment installed with two bed sensors 22, two chair sensors 24, seven motion sensors 20, two of emergency pull cord 30 and/or push button 32, a system gateway 12, a wearable sensor bracelet 36, and an alert light 18.

Again, the wearable sensor bracelet 36 can be substituted with another form of wearable device such as, for example, a sensor-embedded jewelry and a keyring sensor.

From the examples illustrated in FIGS. 8A and 8B, it demonstrates flexibility of the present system to scale up/down according to the needs of the senior residents in a confined area and the area which a group of caregivers/medical practitioners needs to monitor and provide cares. The present system is modifiable in terms of the number of sensors/condition selectable switches, corresponding alert signal generator devices and paired pairing devices, receiving devices, and the internal and/or external devices controlling and monitoring thereof.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

1. An integrated system for monitoring, tracking and recording activities of daily livings (ADLs) and safety and wellness conditions of subjects within the same or different locations of a confined area, comprising:
one or more alert signal generators, each of the alert signal generators receiving signals from one or more corresponding sensors and/or condition selectable switches disposed in where proximal to each of the subjects residing in the confined area for sensing motions, locations, and change in environmental and/or personal conditions of the corresponding subject or alarm signals actively or passively triggered by the subject, and being activated by said signals from the sensors;
one or more corresponding pairing devices physically connected to the one or more alert signal generators for pairing with the corresponding alert signal generators to relay corresponding alert signals of the sensed motion, change in environmental conditions, and/or personal conditions by the corresponding sensors or by the condition selectable switches to a receiving device, and to receive one or more of responses, information, commands, and/or instructions from the receiving device;
one or more receiving devices, each of the receiving devices including at least an alert signal transceiver and a processor, to receive the alert signals from the one or more corresponding pairing devices, directly transmit the alert signals to one or more internal or external devices with respect to the system, or process the alert signals from the one or more pairing devices to generate alert data to transmit to said internal or external devices, receive one or more of signals, responses, information, commands, and/or instructions from the one or more internal or external devices, and relay the one or more of signals, responses, information and/or instructions from the one or more internal or external devices to the one or more signal generators or to other internal or external devices;
said internal or external devices responding to said alert signals or data, and one of the internal or external devices assigning an identity and location data to each of the subjects after receiving the alert signals directly from the one or more pairing devices through the one or more receiving devices or the alert data from the one or more receiving devices such that each set of alert signals or data is attached with said identity and location data assigned by said one of the internal or external devices;
the alert signals or data from said receiving devices and one or more of signals, responses, information and/or instructions from said internal or external devices being timestamped, recorded and stored in an internal or external storage in order for being retrieved and reviewed by caregivers and/or medical practitioners for said subjects when in need thereof.

2. The system of claim 1, wherein the sensors comprise one or more of motion, temperature, pressure, light, and gas detection sensors.

3. The system of claim 1, wherein each of the alert signal generators is physically connected with one of the pairing devices through a pin extended from said pairing device.

4. The system of claim 1, wherein the internal or external devices comprise pager, mobile device, computing device, wearables, server, receiving device other than the first receiving device receiving alert signal from the pairing devices; and wherein the internal or external devices are either physically or wirelessly connected to the receiving device, or both.

5. The system of claim 1, wherein when one of the internal or external devices assigns an identity to one of the pairing devices relaying an alert signal to the receiving device after receiving said alert data including a name and location of the subject under monitoring through the receiving device.

6. The system of claim 1, wherein when one of the internal or external devices sends a confirmation notification to the receiving device regarding an attendance to an event associated with the alert data by a user of the one internal or external device, the receiving device sends said confirmation notification as a confirmation data to other internal or external devices also paired to the receiving device such that other users of said other internal or external devices are notified the event associated with the alert data relayed by the pairing device from the alert signal generator device is being attended.

7. The system of claim 6, wherein the alert data, attendance to the event associated therewith, any feedback signal from the receiving device to the pairing device, change of setting of the alert signal generator device and pairing device by one or more of the internal or external devices are either stored in a corresponding memory of said devices or stored in an external memory, or stored in both memory of said devices and external memory.

8. The system of claim 1, wherein one of the internal or external devices monitors status and changes setting of other internal or external devices directly or through a commonly paired receiving device.

9. A method for monitoring, tracking and recording activities of daily livings (ADLs), safety and wellness conditions of a subject under intensive care by caregivers and/or medical practitioners using the system of claim 1, the method comprising:
disposing a plurality of sensors and/or condition selectable switches in where proximal to the subject residing in a confined area;
connecting said plurality of sensors and/or condition selectable switches to an alert signal generator device;
connecting a corresponding pairing device to said alert signal generator;
pairing said pairing device to said alert signal generator;
connecting said pairing device to a receiving device which is connected to a first internal or external device which monitors connection and operational statuses of said sensors, condition selectable switches, alert signal generator, and/or pairing device;
said receiving device further connecting to a second and/or subsequent internal or external devices being used by said caregivers and/or medical practitioners;
when one of the sensors/condition selectable switches sends a signal to the alert signal generator due to a change in motion, temperature, proximity, moisture, pressure, gas level, and any other environmental or personal data, the paired pairing device sends an alert signal data to the receiving device before relaying thereof to different internal or external devices connected to said receiving device;
once the second or subsequent internal or external device sends a confirmation notification or message regarding attendance to an event associated with the alert signal data to the receiving device, other internal or external devices will receive a message from the receiving device that the event is attended;
one or more of the alert signal data, change of setting in said sensors, condition selectable switches, alert signal generator device, corresponding pairing device, second and/or subsequent internal or external devices, the attendances of different events associated with the alert signal data, feedback signal from one or more internal or external devices to the change of setting in different devices of the system and/or to the attendances of different events is/are recorded and stored in either local or external memory of the system which is accessible and retrievable by the first internal or external device.

10. The method of claim 9, wherein the sensors comprise one or more of motion, temperature, pressure, light, and gas detection sensors.

11. The method of claim 9, wherein each of the alert signal generators is physically connected with one of the pairing devices through a pin extended from said pairing device.

12. The method of claim 9, wherein the internal or external devices comprise pager, mobile device, computing device, wearables, server, receiving device other than the first receiving device receiving alert signal from the pairing devices; and wherein the internal or external devices are either physically or wirelessly connected to the receiving device, or both.

13. The method of claim 9, wherein when one of the internal or external devices assigns an identity to one of the pairing devices relaying an alert signal to the receiving device after receiving said alert data including a name and location of the subject under monitoring through the receiving device.

14. The method of claim 9, wherein when one of the internal or external devices sends a confirmation notification to the receiving device regarding an attendance to an event associated with the alert data by a user of the one internal or external device, the receiving device sends said confirmation notification as a confirmation data to other internal or external devices also paired to the receiving device such that other users of said other internal or external devices are notified the event associated with the alert data relayed by the pairing device from the alert signal generator device is being attended.

15. The method of claim 14, wherein the alert data, attendance to the event associated therewith, any feedback signal from the receiving device to the pairing device, change of setting of the alert signal generator device and pairing device by one or more of the internal or external devices are either stored in a corresponding memory of said devices or stored in an external memory, or stored in both memory of said devices and external memory.

16. The method of claim 9, wherein one of the internal or external devices monitors status and changes setting of other internal or external devices directly or through a commonly paired receiving device.
